(19) United States
(12) Patent Application Publication
(10) Pub. No.: US 2015/0073523 A1
(43) Pub. Date: Mar. 12, 2015

(54) ENDOLEAK ISOLATION SLEEVES AND METHODS OF USE

(71) Applicant: TRIVASCULAR, INC., SANTA ROSA, CA (US)

(72) Inventor: Michael V. CHOBOTOV, Santa Rosa, CA (US)

(73) Assignee: TRIVASCULAR, INC., Santa Rosa, CA (US)

(21) Appl. No.: 14/481,834

(22) Filed: Sep. 9, 2014

Related U.S. Application Data
(60) Provisional application No. 61/875,881, filed on Sep. 10, 2013.

(51) Int. Cl. A61F 2/07; A61L 33/00; A61L 31/04; A61F 2/848; A61F 2/958

(52) U.S. Cl. CPC A61F 2/07; A61F 2/848; A61F 2/958; A61L 31/048; A61L 33/00; A61F 2002/077; A61F 2002/8483; A61F 2002/9583; A61F 2002/072
USPC 623/1.11; 623/1.13

(57) ABSTRACT

Devices and methods for reducing or eliminating endoleaks by isolating feeder vessels from an aneurysm of a patient. In some cases, a tubular isolation sleeve may be deployed in a patient's aneurysm prior to deployment of an endograft such as a modular bifurcated endograft.

### RELATED APPLICATIONS

This application claims priority under 35 U.S.C. 119(e) from U.S. Provisional Patent Application Ser. No. 61/875,881, filed Sep. 10, 2013, by Michael Chobotov, titled “Endoleak Isolation Sleeves and Methods of Use”, which is incorporated by reference herein in its entirety.

### BACKGROUND

An aneurysm is a medical condition indicated generally by an expansion and weakening of the wall of an artery of a patient. Aneurysms can develop at various sites within a patient's body. Thoracic aortic aneurysms (TAAs) or abdominal aortic aneurysms (AAAs) are manifested by an expansion and weakening of the aorta which is a serious and life threatening condition for which intervention is generally indicated. Existing methods of treating aneurysms include invasive surgical procedures with graft replacement of the affected vessel or body lumen or reinforcement of the vessel with a graft.

Surgical procedures to treat aortic aneurysms can have relatively high morbidity and mortality rates due to the risk factors inherent to surgical repair of this disease as well as long hospital stays and painful recoveries. This is especially true for surgical repair of TAAs, which is generally regarded as involving higher risk and more difficulty when compared to surgical repair of AAAs. An example of a surgical procedure involving repair of a AAA is described in a book titled Surgical Treatment of Aortic Aneurysms by Denton A. Cooley, M.D., published in 1986 by W. B. Saunders Company.

Due to the inherent risks and complexities of surgical repair of aortic aneurysms, endovascular repair has become a widely-used alternative therapy, most notably in treating AAAs. Early work in this field is exemplified by Lawrence, Jr. et al. in “Percutaneous Endovascular Graft: Experimental Evaluation”, Radiology (May 1987) and by Mirich et al. in “Percutaneously Placed Endovascular Grafts for Aortic Aneurysms: Feasibility Study,” Radiology (March 1989). Commercially available endoprostheses for the endovascular treatment of AAAs include the AneuRx® stent graft manufactured by Medtronic, Inc. of Minneapolis, Minn., the Zenith® stent graft system sold by Cook, Inc. of Bloomington, Ind., the PowerLink® stent-graft system manufactured by Endologix, Inc. of Irvine, Calif., and the Excluder® stent graft system manufactured by W.L. Gore & Associates, Inc. of Newark, Del.. A commercially available stent graft for the treatment of TAAs is the TAG™ system manufactured by W.L. Gore & Associates, Inc.

Even after successful deployment of an endoprosthesis, continued pressurization of an abdominal aortic aneurysm sac following exclusion using an endograft can contribute to sac enlargement in some instances. In cases where sac enlargement occurs, persistent sac inflow of blood following flow reversal in a patent inferior mesenteric artery (IMA) or lumbar arteries may occur in some patients. Some of these patients may ultimately require a secondary procedure to occlude such a type II endoleak. What have been needed are devices and methods for preventing such endoleaks or reducing the negative effects thereof.

### SUMMARY

Some embodiments of a self-expanding tubular isolation sleeve for treatment of an aneurysm and reduction of endoleaks, may include a self-expanding resilient frame. The self-expanding resilient frame may include one or more resilient strands formed into a tubular structure that is configured to expand from a radially constrained state to a radially expanded state and conform to an irregular morphology of an abdominal aortic aneurysm. The tubular isolation sleeve may also include at least one tubular layer of thin flexible sheet material disposed on the resilient frame, the flexible sheet material optionally having an outside surface that is configured to seal against an inner wall of an aneurysm and isolate a feeder vessel of the aneurysm.

Some embodiments of a method of treating an aneurysm include advancing a tubular isolation sleeve delivery system within a patient's vasculature to a treatment site that includes an aneurysm having a feeder vessel. The tubular isolation sleeve may then be deployed from the delivery system within the aneurysm such that an outer surface of the tubular isolation sleeve seals off the feeder vessel from an interior volume of the aneurysm. Thereafter, an endograft may be deployed at the aneurysm and within an interior lumen of the deployed tubular isolation sleeve.

Some embodiments of a self-expanding tubular isolation sleeve for treatment of an aneurysm and reduction of endoleaks include a self-expanding resilient frame having one or more resilient strands formed into a tubular structure that is configured to expand from a radially constrained state to a radially expanded state. The self-expanding resilient frame may also be configured to conform to an irregular morphology of an abdominal aortic aneurysm. The tubular isolation sleeve may also have at least one tubular layer of thin flexible sheet material disposed on the resilient frame, the thin flexible sheet material having an outside surface that is configured to seal against an inner wall of an aneurysm and isolate a feeder vessel of the aneurysm. In addition, the tubular isolation sleeve may have a fusiform configuration wherein an outer profile of the tubular isolation sleeve in a relaxed unconstrained state is configured to roughly approximate a profile of an interior surface of a typical abdominal aortic aneurysm and wherein the outer profile includes a proximal reduced transverse dimension section at a proximal end thereof, a distal reduced transverse dimension section at a distal end thereof and an enlarged center section of greater transverse dimension than the proximal and distal reduced transverse dimension sections.

Some embodiments of a method of treating an aneurysm include advancing a tubular isolation sleeve delivery system within a patient's vasculature to a treatment site that includes an aneurysm having a feeder vessel and deploying a tubular isolation sleeve from the tubular isolation sleeve delivery system within the aneurysm such that an outer surface of the tubular isolation sleeve interrupts blood flow from the feeder vessel to the aneurysm. Thereafter, an endograft may be deployed at the aneurysm and within an interior lumen of the deployed tubular isolation sleeve.

### BRIEF DESCRIPTION OF THE DRAWINGS

FIG. 1 is an elevation view of an embodiment of a tubular isolation sleeve in a radially constrained configuration.

FIG. 2 is a transverse cross section view of the tubular isolation sleeve embodiment of FIG. 1 taken along lines 2-2 of FIG. 1.

FIG. 3 is a transverse cross section view an embodiment of a tubular isolation sleeve having two inner layers of flexible sheet material and a single outer layer of flexible sheet material.

FIG. 4 is a transverse cross section view of an embodiment of a tubular isolation sleeve having two outer layers of flexible sheet material and a single inner layer of flexible sheet material.

FIG. 5 is an elevation view of the tubular isolation sleeve embodiment of FIG. 1 in a radially expanded and relaxed state.

FIG. 5A is an elevation view of a tubular isolation sleeve embodiment having a fusiform configuration in a radially expanded and relaxed state.

FIG. 6 shows a distal section of a tubular isolation sleeve delivery system embodiment disposed over a guidewire embodiment and within a patient's abdominal aorta at a treatment site that includes an abdominal aortic aneurysm.

FIG. 7 shows the tubular isolation sleeve delivery system embodiment of FIG. 6 with an outer sheath of the delivery system partially retracted and the tubular isolation sleeve embodiment partially expanded in a radial direction and partially deployed within the aneurysm.

FIG. 8 shows the tubular isolation sleeve embodiment of FIG. 7 fully deployed within the aneurysm so as to fluidly isolate an IMA feeder vessel from an interior volume of aneurysm.

FIG. 9 shows a modular bifurcated endograft embodiment fully deployed within the aneurysm and within an interior lumen of the tubular isolation sleeve.

The drawings illustrate embodiments of the invention and are not limiting. For clarity and ease of illustration, the drawings are not made to scale and, in some instances, various aspects may be shown exaggerated or enlarged to facilitate an understanding of particular embodiments.

### DETAILED DESCRIPTION

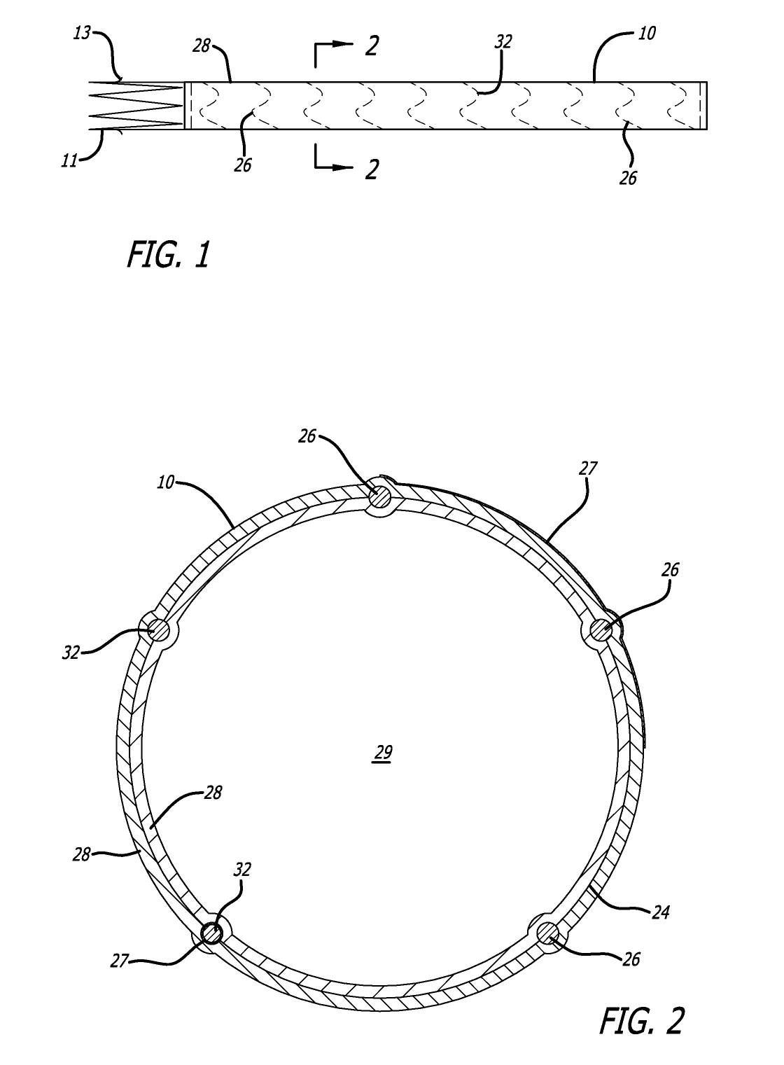

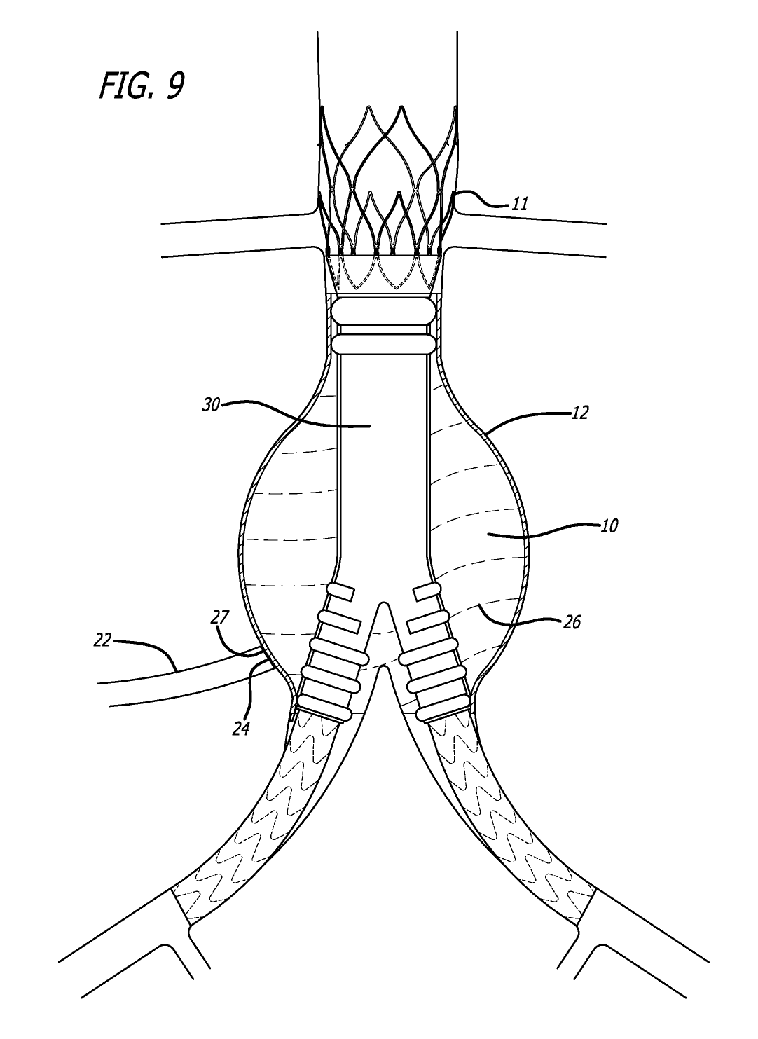

As discussed above, continued pressurization of an abdominal aortic aneurysm sac following exclusion using an endograft can contribute to sac enlargement in some instances. In cases where sac enlargement occurs, persistent sac inflow of blood following flow reversal in patent IMA or lumbar arteries which may be acting as feeder arteries, may occur in some patients. Some such patients ultimately may receive a secondary procedure to occlude such a type II endoleak caused by feeder vessels or other causes. As a preventative measure, or for any other suitable indication, a tubular isolation sleeve, such as the tubular isolation sleeve embodiment 10 shown in FIG. 1, may be deployed within an aneurysm sac, such as the aneurysm sac 20 shown in FIG. 6. Such tubular isolation sleeve embodiments 10 may be percutaneously delivered at a treatment site (such as an aneurysm 12) in order to isolate or seal off one or more feeder vessels prior to deployment of a standard or otherwise commercially available endograft. An example of such a modular bifurcated endograft 30 is shown in a deployed state in FIG. 9 with the endograft 30 deployed within an inner lumen of the tubular isolation sleeve embodiment 10. In some cases, the tubular isolation sleeve 10 may be deployed prior to deployment of the endograft 30, with both the tubular isolation sleeve 10 and endograft 30 being deployed during a single procedure for treating a patient.

Some tubular isolation sleeve embodiments 10 may be configured to have a self-expanding configuration and self-expand in an outward radial direction from a radially constrained state to a radially expanded state. In some cases, such outward radial expansion may be facilitated by the use of superelastic materials for the strands 26. It should be noted that although the tubular isolation sleeve 10 is allowed to expand radially upon deployment, the tubular isolation sleeve 10 in many cases may not expand to a fully expanded state. That is, after deployment, some or all of the tubular isolation sleeve 10 may remain constrained by an inner surface of the aneurysm in which it is deployed. In addition, some tubular isolation sleeve embodiments 10 may have a non-self-expanding arrangement and be expandable by suitable devices configured to exert an outward radial force on the wall of the tubular isolation sleeve from within, such as an inflatable balloon.

An optional external anchoring stent 11 that may or may not include tissue engaging barbs 13 may be secured to and extend proximally from a proximal end of the tubular isolation sleeve 10. Such an optional anchoring stent may include a self-expanding configuration which may also be facilitated by the use of superelastic materials such as NiTi. The barbs 13 may have sharpened tips which are disposed at an angle with respect to the anchoring stent to engage tissue disposed about the anchoring stent 11 in a deployed state and prevent distal migration of the tubular isolation sleeve 10 once deployed. In some cases, the barbs may be generally oriented in a distal direction. Anchoring stent embodiments are discussed in more detail in the commonly owned patent applications which are incorporated by reference herein. Any suitable stent configuration discussed in these incorporated applications may be used as the anchoring stent 11.

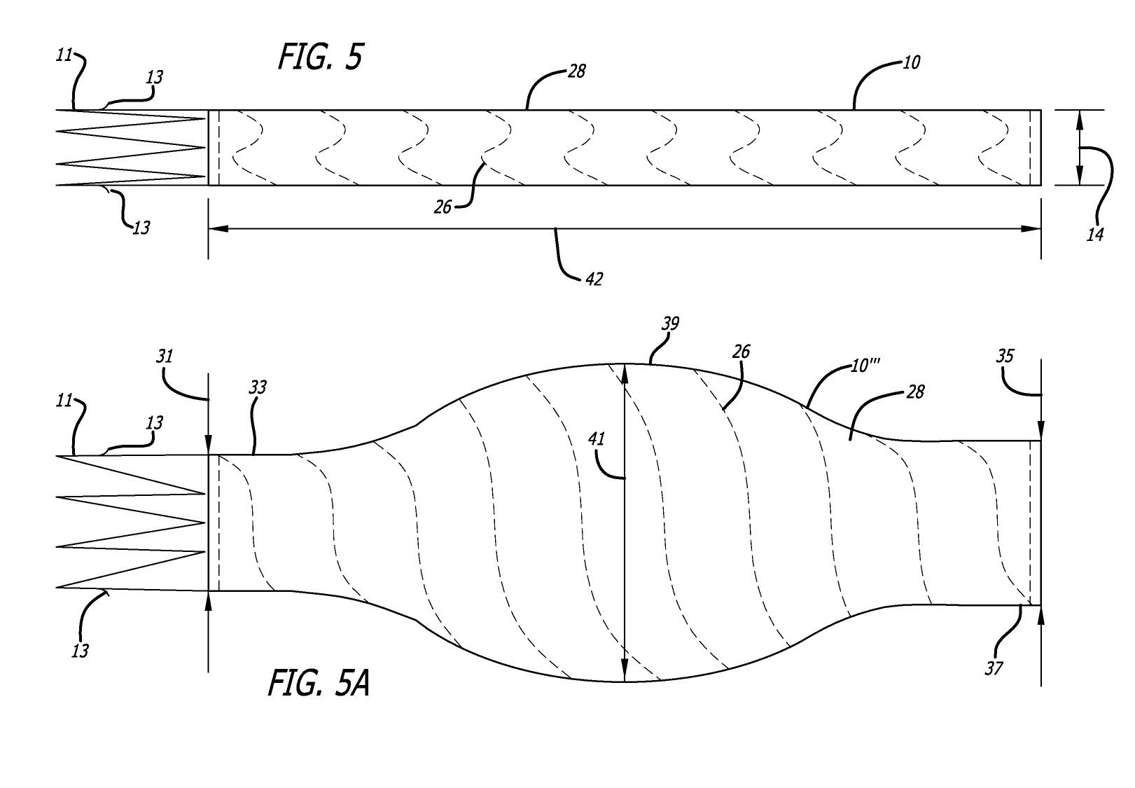

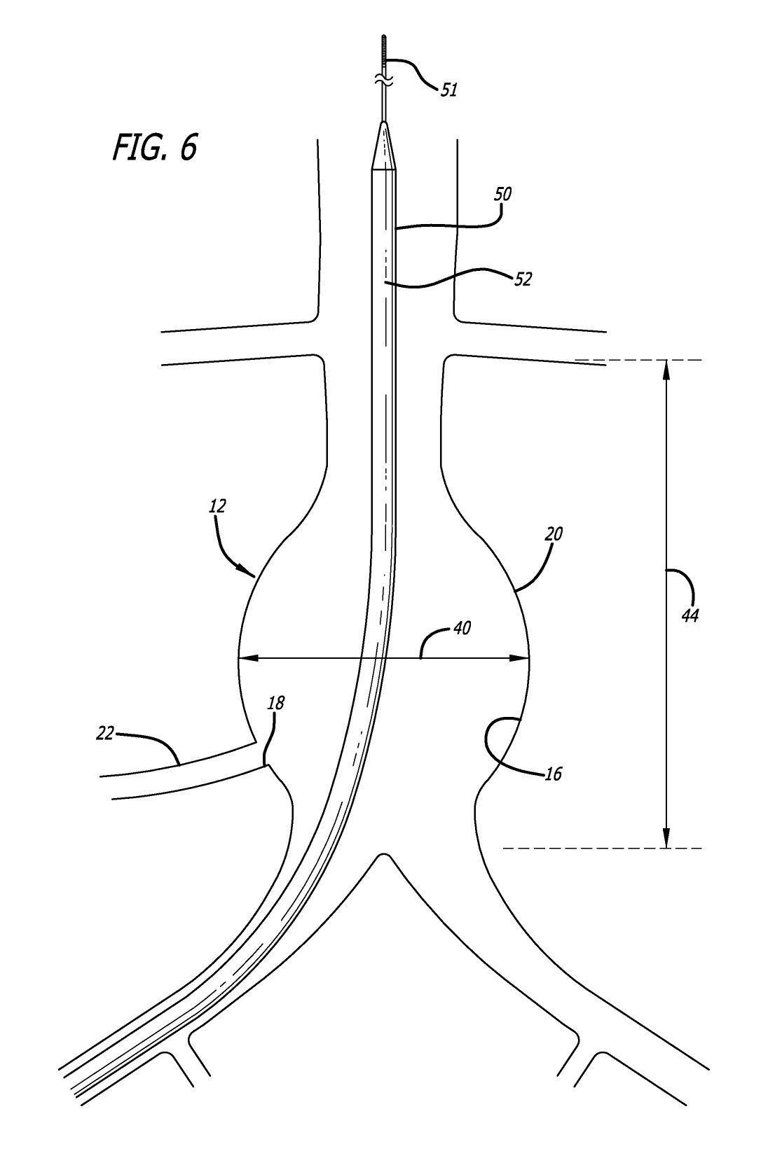

Tubular isolation sleeve embodiments 10 may typically be larger in a transverse dimension 14 in an expanded state (as shown in FIG. 5) than in a radially constrained state (as shown in FIG. 1). It should be noted that for embodiments discussed herein, reference to a transverse dimension 14 may include a transverse diameter for embodiments having a round transverse cross section. The tubular isolation sleeve 10 may self-expand from the radially constrained state to a radially expanded state such that an outer surface of the tubular isolation sleeve 10 contacts an inner wall surface 16 of a body lumen such as an inner surface of an infrarenal aortic lumen or abdominal aorta. In such a procedure, the ostium 18 (see FIG. 6) of any potential type II feeder vessels, such as an IMA 22, communicating with the aneurysm sac 20 may be isolated, sealed off or otherwise occluded by a wall layer 24 (see FIG. 1) of the tubular isolation sleeve 10. Suitable isolation of these type II feeder vessels may include a complete or nearly complete sealing of the outer wall 24 of the tubular isolation sleeve 10 to the inner wall surface 16. However, suitable isolation may also include interrupting a flow of blood from the feeder vessels (without a complete or nearly complete seal) which is sufficient to promote thrombosis within the feeder vessel. Such isolation of the feeder vessel 22 may then promote thrombosis of blood within these feeder vessels and occlude these feeder vessels so as to preclude them from causing type II endoleaks at the treatment site 12.

Some suitable tubular isolation sleeve embodiments 10 (particularly those that have a self-expanding configuration) may be constructed from a combination of high strength resilient strands 26, including superelastic strands 26 made from a material such as a Nitinol alloy (NiTi) or the like. In addition to the resilient strands 26, the construction of some tubular isolation sleeve embodiments 10 may include a thin flexible sheet material 28 disposed in a tubular configuration. The thin flexible sheet material 28 may be configured to restrict or prevent the passage of body fluids such as blood therethrough and may include polytetrafluoroethylene (PTFE), nylon materials such as Dacron® and the like.

As discussed above, it may be desirable to interrupt flow of feeder vessels 22 or occlude feeder vessels 22 by causing thrombosis of blood within an inner lumen of feeder vessels 22. As such, in some instances, it may be desirable to include a thrombogenic or clotting agent 27 on an outside surface of any of the tubular isolation sleeve embodiments discussed herein. In particular, it may be desirable to include a clotting agent 27 such as thrombin or the like on an outside surface of the resilient strands 26 or the thin flexible sheet material 28 of the tubular isolation sleeve 10. In some cases, a clotting agent 27 may be disposed on an entire surface of the resilient strands 26, flexible sheet material 28, or both the resilient strands 26 and flexible sheet material 28. In other cases, the clotting agent 27 may be disposed only on selected a selected portion or portions of the outside surface of either or both the resilient strands 26 or flexible sheet material 28.

The selected portion or portions to be coated with clotting agent 27 may include those portions of the tubular isolation sleeve 10 that are configured to cover or be near an ostium of a feeder vessel 27 when the tubular isolation sleeve 10 is deployed. It should also be noted that although the use of a clotting agent 27 has been discussed in terms of coating a portion or portions of the tubular isolation sleeve 10, the clotting agent 27 may be disposed on any portion of the tubular isolation sleeve 10 where it will eventually be in fluid communication with an outside surface of a desired portion of the tubular isolation sleeve 10. Thus, the clotting agent 27 need not necessarily be coated on an outside surface and may be disposed within the structure of the tubular isolation sleeve 10 so long as fluid such as blood may carry the clotting agent 27 to a position outwardly adjacent the outer surface of the tubular isolation sleeve 10.

For some embodiments, the resilient strands 26 may include a fine gauge wire having a transverse dimension of about 0.006 inches to about 0.010 inches and such wire may be formed into a stent-like structure over a tool such as mandrel in a helical pattern (as shown in the embodiment 10 of FIG. 1) with periodic undulations such as is shown and discussed with regard to the endograft extensions discussed in U.S. Patent Publication No. 2009/0099649, filed Oct. 3, 2008, titled “Modular Vascular Graft for Low Profile Percutaneous Delivery”, which is incorporated by reference herein in its entirety.

The resilient strands 26 so formed into a generally tubular stent-like shape may also be encapsulated by one or more layers of the thin flexible sheet material 28 (such as PTFE or ePTFE film) so as to form a tubular structure having a central lumen 29 with self-expanding walls 24. The self-expanding walls 24 may be resistant to a flow of body fluids such as blood from a location inside the central lumen 29 to a location outside the tubular isolation sleeve 10. That is, the self-expanding walls 24 may be impervious or substantially impervious to a flow of liquids such as blood therethrough. In some cases, the tubular isolation sleeve 10 may include about 1 layer to about 3 layers of thin flexible sheet material 28 disposed about the stent-like tubular structure (which may also be referred to as a frame 32) of the resilient strand or strands 26.

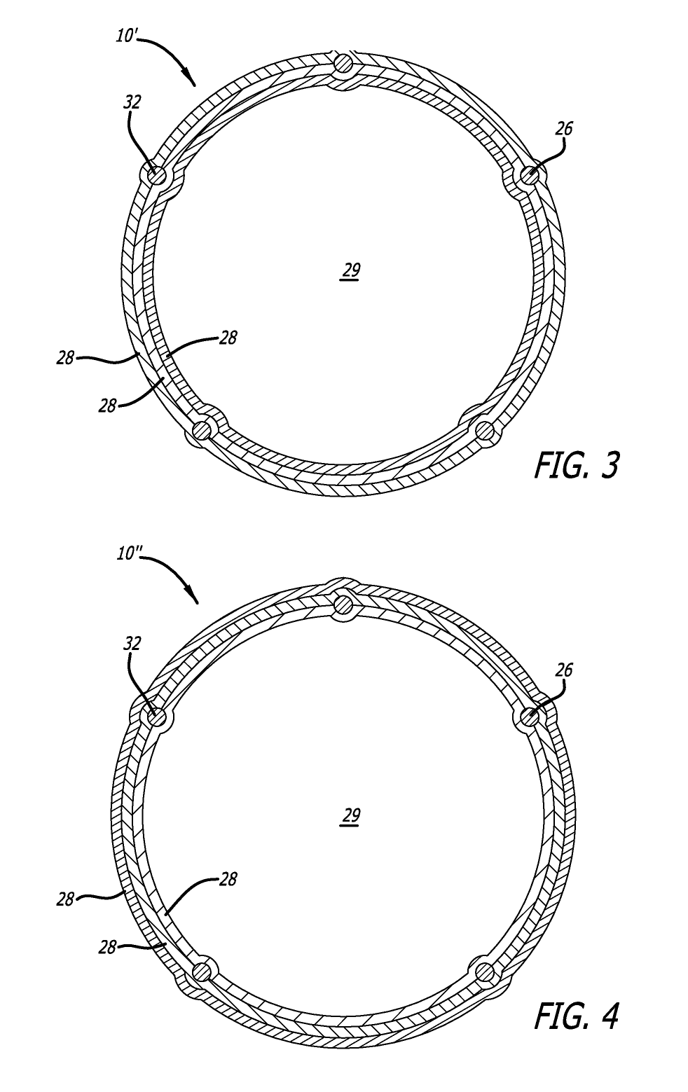

FIG. 2 shows the tubular isolation sleeve embodiment 10 having a single outer layer of flexible sheet material 28 disposed on an outside surface of the frame 32 and a single inner layer of flexible sheet material 28 disposed on an inside surface of the frame 32. FIG. 3 shows a tubular isolation sleeve embodiment 10′ having two inner layers of flexible sheet material 28 disposed on an inside surface of the frame 32 and a single outer layer of flexible sheet material 28 disposed on an outside surface of the frame 32. FIG. 4 shows a tubular isolation sleeve embodiment 10″ having two outer layers of flexible sheet material 28 disposed on an outside surface of the frame 32 and a single inner layer of flexible sheet material 28 disposed on an inside surface of the frame 32.

For some embodiments, the transverse dimension 14 (FIG. 5) of the tubular isolation sleeve 10 in an unconstrained state (which may also be referred to as a free state) may be configured to be somewhat greater than a nominal internal transverse dimension 40 (FIG. 6) of a lumen of an aneurysm sac 20 to be treated. For example, if an abdominal aortic aneurysm sac 20 that is to be treated has an inner lumen with a maximum transverse dimension 40 of about 6 cm, a tubular isolation sleeve embodiment 10 having a nominal transverse dimension 14 (when in an unconstrained state) of about 7 cm to about 8 cm may be sufficiently oversized to ensure stability of the tubular isolation sleeve 10 once deployed within the aneurysm sac 20. In some cases, tubular isolation sleeves 10 may have a transverse dimension 14 in an unconstrained free state that is up to about 80% oversized relative to an inner transverse dimension of the aneurysm sac that is to be treated. As aneurysms are typically non-uniform in their inner transverse dimension, the oversizing of the tubular isolation sleeve 10 may be configured in a segmented fashion whereby one or more axial segments or sections of tubular isolation sleeve may each be oversized by a desired amount relative to a corresponding axial section of an aneurysm to be treated.

It may be important in some cases for tubular isolation sleeve embodiments 10 to remain in a stable position with respect to the aneurysm sac 20 for the time period between deployment of the tubular isolation sleeve 10 and subsequent deployment of an endograft 30 at the same treatment site 12. In some cases, tubular isolation sleeve embodiments 10 may be sized with regard to an axial length 42 (see FIG. 5) of the tubular isolation sleeve 10 to span an axial length 44 (see FIG. 6) from a neck of the aneurysm being treated to the aortic bifurcation of the iliac arteries. For some patients, this distance 44 may be about 8 cm to about 12 cm. Thus, for some embodiments, the tubular isolation sleeve 10 may have an axial length 42 of about 8 cm to about 12 cm. In some instances, it may only be desirable to stock a small number of sizes of tubular isolation sleeves 10 (axial length 42 and transverse dimension 14) in an operating room or catheter lab in order to treat the typical range of abdominal aortic aneurysm morphologies.

Some embodiments of a tubular isolation sleeve may also include a fusiform type shape in the relaxed expanded state with a reduced transverse dimension at a proximal end and distal end thereof. FIG. 5A shows such an embodiment wherein the outer profile of the tubular isolation sleeve 10″′ is configured to roughly approximate a profile of an interior surface of a typical abdominal aortic aneurysm. Such a profile may be useful in order for an outer surface of the tubular isolation sleeve 10 to conform to and approximate the inner surface of an aneurysm 20 being treated such that the flow of blood from feeder vessels 22 may be sufficiently interrupted or otherwise sealed off from the lumen of the host artery to prevent endoleaks. This profile as shown includes a reduced transverse dimension 31 at a proximal reduced transverse dimension section 33 thereof and a reduced transverse dimension 35 at a distal reduced transverse dimension section 37 thereof. The outer profile of the tubular isolation sleeve 10″′ may have a smooth and continuous curve from an enlarged center section 39 of greater transverse dimension relative to the reduced transverse dimension sections 31 and 35 at the proximal end and distal end of the tubular isolation sleeve 10″′. The enlarged center section 39 has a transverse dimension 41 that may be up to about 4 times the transverse dimension of reduced diameter section 31 of the proximal end 33, the reduced diameter section 35 of the distal end 37, or both the reduced diameter section 31 and reduced diameter section 35. In some cases, the enlarged center section 39 may have a transverse dimension 41 that is about 1 times to about 4 times the transverse dimension of reduced diameter section 31 of the proximal end 33, the reduced diameter section 35 of the distal end 37, or both the reduced diameter section 31 and reduced diameter section 35. In some cases, the enlarged center section 39 may have a transverse dimension 41 that is about 1.4 times to about 3 times the transverse dimension of reduced diameter section 31 of the proximal end 33, the reduced diameter section 35 of the distal end 37, or both the reduced diameter section 31 and reduced diameter section 35.

The features, dimensions and materials of fusiform tubular isolation sleeve embodiments 10″′ may otherwise be the same as those of the non-fusiform tubular isolation sleeve embodiments 10 of FIG. 1. In particular, the sizing of the tubular isolation sleeve 10″′ may be oversized in its expanded relaxed state with respect to the transverse dimensions of the inner surface of the sac of the aneurysm being treated 20 in ratios which are the same as or similar to those of the other tubular isolation sleeve embodiments discussed herein. The tubular isolation sleeve embodiment 10″′ may have transverse dimensions 31, 35 and 41 in an unconstrained relaxed state which are up to about 80% oversized relative to respective transverse dimensions of an aneurysm to be treated.

The helical pattern of the resilient wires 26 of some tubular isolation sleeve embodiments 10 may allow for adjustment of axial length 42 (FIG. 5) of the tubular isolation sleeve 10 such that a small number of device configurations of varying axial lengths may be capable of treating most anatomies. A suitable self-expanding tubular isolation sleeve embodiment 10 may also include a plurality of distinct and separate rings (not shown) of resilient strand material 26 that would also achieve a similar result of allowing for adjustment of axial length 42. In some cases, it may only be desirable to use tubular isolation sleeve embodiments 10 having about 1 to about 3 different axial lengths 42 in order to treat a large percentage or majority of patient's requiring such a self-expanding tubular isolation sleeve 10. In some cases, it may also not be necessary for a self-expanding tubular isolation sleeve embodiment 10 (once deployed) to cover the entire region or inner surface 16 of the aorta 12 extending from the inferior renal arteries to the bifurcation at the iliac arteries. In such cases, it may only be desirable that potential sac feeder vessels (such as IMA 22) be covered or otherwise isolated by the wall 24 of the tubular isolation sleeve 10 which is disposed over such vessels 22 and seals them from the interior lumen or volume of the parent aorta 12. Such arrangements may be determined by pre-operative imaging. The subsequently deployed endograft 30 may be delivered, positioned and deployed in a normal manner, effectively trapping the deployed self-expanding tubular isolation sleeve 10 in the excluded aneurysm sac 12 as shown in FIG. 9.

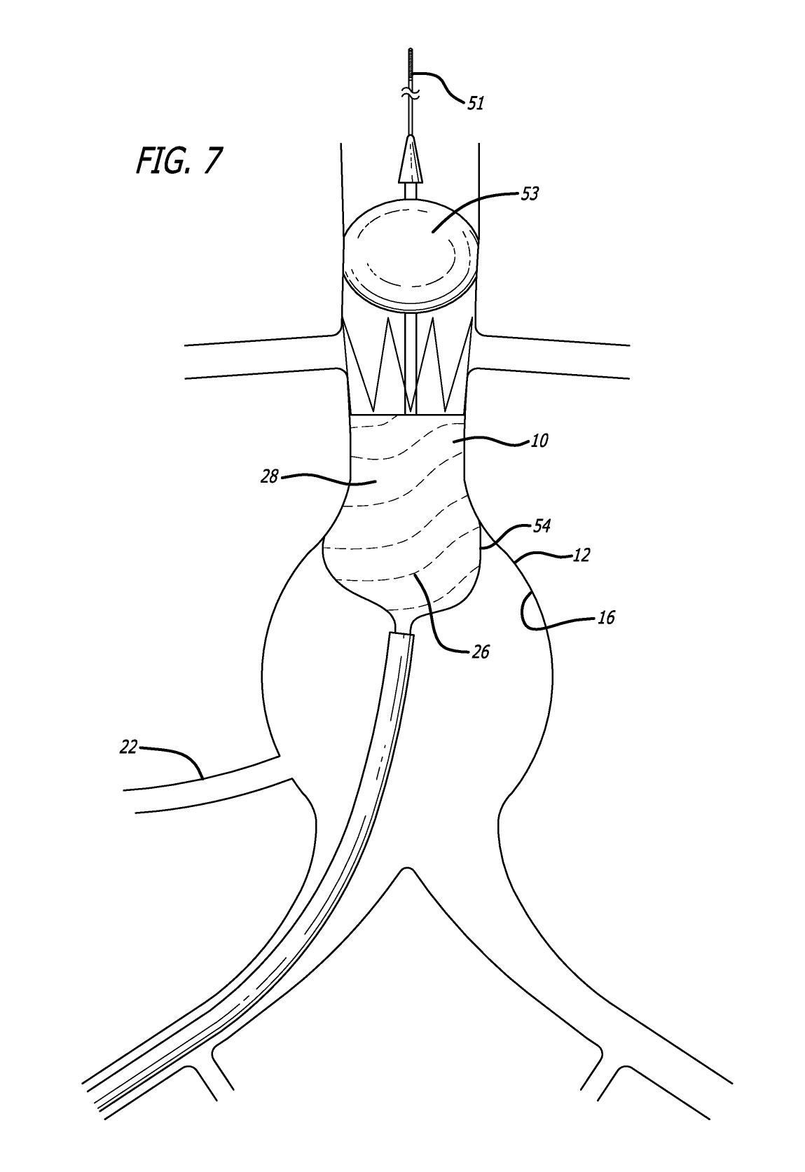

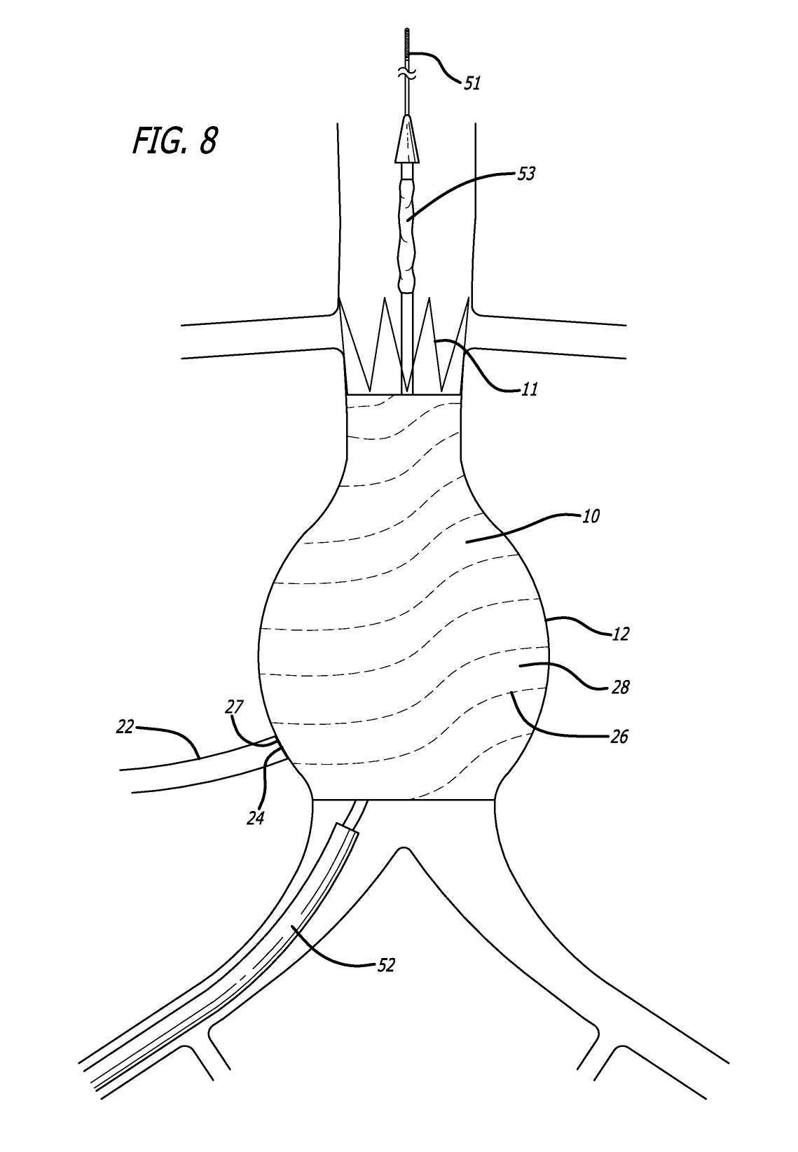

Embodiments of tubular isolation sleeves 10 may be compatible with any or most endograft embodiments, and may be deployed in a pull-back sheath type delivery system such as shown in U.S. Patent Publication No. 2006/0009833, filed Aug. 15, 2005, titled “Delivery System and Method for Bifurcated Graft”, which is incorporated by reference herein in its entirety. Some embodiments of a method of treating an aneurysm 12 may include advancing a tubular isolation sleeve delivery system 50 over a guidewire 51 within a patient's vasculature to a treatment site 12 (FIG. 6). A particularly suitable treatment site may include an aneurysm 12 having a feeder vessel such as IMA 22 or the like. The delivery system 50 includes a tubular isolation sleeve 10 in a radially constrained state with an inner surface of an outer sheath 52 exerting an inward radial constraining force on an outer surface of the tubular isolation sleeve 10. The tubular isolation sleeve 10 may then be deployed within the aneurysm 12 by retracting the outer sheath 52 of the delivery system 50 so as to expose the tubular isolation sleeve 10, release the radial constraining force of the inner surface of the outer sheath 52, and allow self-expansion of the tubular isolation sleeve 10 to occur as the outer sheath 52 is retracted (FIG. 7). The tubular isolation sleeve 10 may be allowed to self-expand towards the inner surface 16 of the aneurysm such that an outer surface 54 of the tubular isolation sleeve 10 contacts the inner surface of the aneurysm 12 and eventually seals off the feeder vessel 22 from an interior volume of the aneurysm 12 (FIG. 8). As discussed above, once the tubular isolation sleeve 10 is deployed, blood flow from feeder vessels 22 may be interrupted such that the ostium 18 (see FIG. 6) of any potential type II feeder vessels, such as an IMA 22, communicating with the aneurysm sac 20 may be isolated, sealed off or otherwise occluded by a wall layer 24 (see FIG. 1) of the tubular isolation sleeve 10. Suitable isolation of these type II feeder vessels 22 may include approximating an outer surface of the tubular isolation sleeve 10 to the ostium 18 of a feeder vessel 22 so as to completely or nearly completely seal the outer wall 24 of the tubular isolation sleeve 10 to the inner wall surface 16 of the aneurysm around the ostium 18. Suitable treatment of feeder vessels 22 may also include interrupting a flow of blood from the feeder vessels (without a complete or nearly complete seal) which is sufficient to promote thrombosis within the feeder vessel 22. Such isolation of the feeder vessel 22 may then promote thrombosis of blood within these feeder vessels and occlude these feeder vessels so as to, e.g., preclude them from causing type II endoleaks at the treatment site 12. Thereafter, an endograft 30 may be deployed at the aneurysm 12 and within an interior lumen of the deployed tubular isolation sleeve 10.

In some cases, deploying the tubular isolation sleeve 10 may include expansion in an outward radial direction of the optional anchoring stent 11 so as to contact the inner wall surface 16. In some cases, the tubular isolation sleeve delivery system may include an optional inflatable radially expanding balloon 53 and deployment of the tubular isolation sleeve 10 may include inflation of the optional inflatable radially expanding balloon 53 (FIG. 7). The inflatable balloon 53 may be disposed on the delivery system 50 proximal of the tubular isolation sleeve 10 and be inflated in a position at the delivery site which is proximal to the position of the tubular isolation sleeve 10. The optional inflatable balloon 53 may be used to slow or stop the flow of blood through a parent vessel of the aneurysm 12 and/or through the aneurysm 12 in order to allow the tubular isolation sleeve 10 to deploy without interference from the turbulence of flowing blood. This may be particularly desirable for deployment of a tubular isolation sleeve embodiments 10 having very thin walls and light gauge frames 32 or embodiments 10 without proximal anchor stents 11. In some cases, deploying the endograft 30 may include deploying a bifurcated endograft embodiment 30 such as the endograft embodiment shown in FIG. 9. The bifurcated endograft, or portions thereof, may also be deployed within an inner lumen of the already deployed tubular isolation sleeve 10.

In some cases, the tubular isolation sleeve embodiments 10 discussed herein may be useful for treating an aneurysm 12 which has ruptured. During such an emergency procedure, the tubular isolation sleeve 10 may be deployed prior to deployment of a standard endograft 30. Such deployment of the tubular isolation sleeve 10 may allow a surgical team sufficient time to deploy the standard endograft 30 without concern about patient blood loss due to the rupture of the aneurysm 12. Use of the tubular isolation sleeve 10 prior to deployment of a standard endograft 30 may be useful in that such a deployment of the tubular isolation sleeve 10 may be a quick and simple deployment procedure relative to the time and complexity of the deployment of a standard endograft 30, particularly a multi-component bifurcated endograft 30.

The entirety of each patent, patent application, publication and document referenced herein hereby is incorporated by reference. Citation of the above patents, patent applications, publications and documents is not an admission that any of the foregoing is pertinent prior art, nor does it constitute any admission as to the contents or date of these publications or documents.

Modifications may be made to the foregoing without departing from the basic aspects of the invention. Although embodiments of the invention have been described in substantial detail with reference to one or more specific embodiments, those of ordinary skill in the art will recognize that changes may be made to the embodiments specifically disclosed in this application, yet these modifications and improvements are within the scope and spirit of the invention.

Embodiments illustratively described herein suitably may be practiced in the absence of any element(s) not specifically disclosed herein. Thus, for example, in each instance herein any of the terms “comprising,” “consisting essentially of,” and “consisting of” may be replaced with either of the other two terms. The terms and expressions which have been employed are used as terms of description and not of limitation, and use of such terms and expressions do not exclude any equivalents of the features shown and described or portions thereof, and various modifications are possible within the scope of the invention claimed. The term “a” or “an” can refer to one of or a plurality of the elements it modifies (e.g., “a reagent” can mean one or more reagents) unless it is contextually clear either one of the elements or more than one of the elements is described. Thus, it should be understood that although embodiments have been specifically disclosed by representative embodiments and optional features, modification and variation of the concepts herein disclosed may be resorted to by those skilled in the art, and such modifications and variations are considered within the scope of this invention.

What is claimed is:

1. A self-expanding tubular isolation sleeve for treatment of an aneurysm and reduction of endoleaks, comprising:
a self-expanding resilient frame including one or more resilient strands formed into a tubular structure that is configured to expand from a radially constrained state to a radially expanded state and conform to an irregular morphology of an abdominal aortic aneurysm;
at least one tubular layer of thin flexible sheet material disposed on the resilient frame which has an outside surface that is configured to seal against an inner wall of an aneurysm and isolate a feeder vessel of the aneurysm; and
a fusiform configuration wherein an outer profile of the tubular isolation sleeve in a relaxed unconstrained state is configured to roughly approximate a profile of an interior surface of a typical abdominal aortic aneurysm and wherein the outer profile includes a proximal reduced transverse dimension section at a proximal end thereof, a distal reduced transverse dimension section at a distal end thereof and an enlarged center section of greater transverse dimension than the proximal and distal reduced transverse dimension sections.

2. The tubular isolation sleeve of claim 1 wherein the enlarged center section has a transverse dimension up to about 4 times the transverse dimension of the proximal reduced diameter section and the distal reduced diameter section.

3. The tubular isolation sleeve of claim 1 wherein the enlarged center section has a transverse dimension that is about 1.4 times to about 3 times the transverse dimension of proximal reduced diameter section and the distal reduced diameter section.

4. The tubular isolation sleeve of claim 1 wherein an axial length of the tubular isolation sleeve is about 8 cm to about 12 cm.

5. The tubular isolation sleeve of claim 1 further comprising a self-expanding anchoring stent secured to and extending proximally from a proximal end of the tubular isolation sleeve.

6. The tubular isolation sleeve of claim 5 wherein the self-expanding anchoring stent further comprises tissue engaging barbs with sharpened tips which are disposed at an angle with respect to the anchoring stent.

7. The tubular isolation sleeve of claim 1 wherein a transverse dimension of the tubular isolation sleeve in an unconstrained free state is up to about 80% oversized relative to an inner transverse dimension of an aneurysm sac that is to be treated.

8. The tubular isolation sleeve of claim 1 wherein the one or more resilient strands are formed into an undulating helical configuration.

9. The tubular isolation sleeve of claim 1 wherein the one or more resilient strands comprise a superelastic material.

10. The tubular isolation sleeve of claim 9 wherein the superelastic material comprises NiTi.

11. The tubular isolation sleeve of claim 1 wherein the tubular layer of flexible sheet material comprises PTFE.

12. The tubular isolation sleeve of claim 1 comprising an outer layer of flexible sheet material disposed on an outer surface of the frame and an inner layer of flexible sheet material disposed on an inner surface of the frame.

13. A method of treating an aneurysm, comprising:
advancing a tubular isolation sleeve delivery system within a patient's vasculature to a treatment site that includes an aneurysm having a feeder vessel;
deploying a tubular isolation sleeve from the tubular isolation sleeve delivery system within the aneurysm such that an outer surface of the tubular isolation sleeve interrupts blood flow from the feeder vessel to the aneurysm; and
subsequently deploying an endograft at the aneurysm and within an interior lumen of the deployed tubular isolation sleeve.

14. The method of claim 13 wherein interrupting blood flow from the feeder vessel comprises sealing the feeder vessel from a sac of the aneurysm.

15. The method of claim 13 wherein interrupting blood flow from the feeder vessel comprises interrupting the blood flow sufficiently to cause thrombosis of blood within the feeder vessel and seal the feeder vessel from a sac of the aneurysm.

16. The method of claim 13 wherein subsequently deploying the endograft at the aneurysm and within the interior lumen of the deployed tubular isolation sleeve comprises so deploying a bifurcated endograft.

17. The method of claim 13 wherein the tubular isolation sleeve comprises a thrombogenic agent and deploying the tubular isolation sleeve comprises positioning the thrombogenic agent of the tubular isolation sleeve in fluid communication with blood within the feeder vessel upon deployment.

18. The method of claim 13 wherein the tubular isolation sleeve delivery system includes an inflatable balloon and further comprising expanding the inflatable balloon proximally of the tubular isolation sleeve to temporarily occlude blood flow in a parent artery of the aneurysm proximal to the tubular isolation sleeve and prior to deploying the tubular isolation sleeve.

19. The method of claim 13 wherein the tubular isolation sleeve is deployed such that the outer surface of the tubular isolation sleeve approximates an ostium of the feeder vessel to interrupt blood flow therefrom.
